(19) United States
(12) Patent Application Publication
(10) Pub. No.: US 2019/0142326 A1
(43) Pub. Date: May 16, 2019

(54) DEVICES, SYSTEMS AND METHODS FOR TESTING OLFACTORY SENSITIVITY

(71) Applicant: INSPIRED LIFE MEDICAL INC., LeSueur, MN (US)

(72) Inventor: Gregory B. Mills, Kansas City, KS (US)

(21) Appl. No.: 16/189,801

(22) Filed: Nov. 13, 2018

Related U.S. Application Data
(60) Provisional application No. 62/585,628, filed on Nov. 14, 2017.
(60) Provisional application No. 62/749,465, filed on Oct. 23, 2018.

(51) Int. Cl. A61B 5/00

(52) U.S. Cl. CPC A61B 5/4011; A61B 5/6819

(57) ABSTRACT

The present system is directed in various embodiments to devices and methods for testing a patient's olfactory capability and/or sensitivity. The devices and methods may be used to test and compare a patient's ability to detect and/or identify an aroma or odorant presented to each nostril. Alternatively, the devices and methods may be used to generally assess, without regard to nostril-to-nostril comparative results, a patient's olfactory ability. The device may comprise a disk that comprises a plurality of odorant compartments that may be progressively advanced as the testing for a subject odorant is completed. The device may comprise a passive airway and an active airway, wherein only one nostril receives aroma-infused air while the other nostril receives atmospheric air. In other cases, the device may present aroma-infused air to both nostrils at the same time.

### CROSS-REFERENCE TO RELATED APPLICATIONS

This application claims the benefit of U.S. Provisional Application Ser. No. 62/585,628, filed Nov. 14, 2017 and entitled METHODS OF DIAGNOSING ALZHEIMER'S DISEASE AND AROMA PRESENTATION DEVICES and U.S. Provisional Application Ser. No. 62/749,465, filed Oct. 23, 2018 and entitled METHOD OF DIAGNOSING LIVING NON-DEMENTED INDIVIDUALS WITH ALZHEIMER'S NEUROPATHOLOGY, the entire contents of each of which are hereby incorporated by reference.

### FIELD OF THE INVENTION

The present invention generally relates to devices, systems and methods for determining olfactory detection thresholds.

### DESCRIPTION OF THE RELATED ART

Aroma testing in the past has been generally related to the overall aroma detection of a person, commonly by naming a particular odor, without particular interest in comparing the relative smelling ability of their nostrils or the type of aromas they could and could not smell. Published articles document that a relatively poor sense of smell in the left nostril, or sensitivity, as compared to the sensitivity of the right nostril may be indicative of early brain damage due to neurological disease. See, e.g., Murphy, et al., “Left hippocampal volume loss in Alzheimer's disease is reflected in performance on odor identification: A structural MRI Study”, Journal of the International Neuropsychological Society, Vol. 9, No. 3, pp 459-471 (2003). This is the case in Alzheimer's Disease (hereinafter AD), but is of clinical significance in the early detection of AD, however, only if the aroma used in the test is a pure aroma for reasons that are discussed further infra.

Another screening methodology takes advantage of the notable deficit of smelling ability in the left nostril as compared to the smelling ability of the right nostril to detect a pure aroma, appears according to previous medical research, to be indicative of early neurological degeneration of the olfactory nerve, specifically as seen in the early onset of AD.

The olfactory nerve is found on the left side of the brain and is not reversed bilaterally as many brain functions are, such as eye sight. Research, including data from autopsies, indicates that degeneration of the olfactory nerve occurs gradually and begins very early in the disease development of AD. Such deterioration may begin years before substantial dementia becomes notable. Thus currently a diagnosis of AD becomes confirmed by existing testing procedures that are generally focused on observation, detection and/or diagnosis of actual dementia. This is problematic in the detection of AD because, inter alia, dementia presents in other non-AD diseases, conditions and/or disorders. Moreover, at such late disease stages, AD is generally not amenable to treatment. Consequently, screening and diagnosis at the earliest stage possible is critical.

Known methods and devices for testing generally olfactory ability and more specifically bilateral differences in olfactory abilities are unsatisfactory for a number of reasons and, therefore, a device and method that is easy to use would be of great value.

Applicant has developed several solutions to the problem of bilateral screening as further described in U.S. patent application Ser. No. 14/282,622, entitled DEVICES, SYSTEMS AND METHODS FOR DETECTING A BILATERAL DIFFERENTIAL IN OLFACTORY THRESHOLD FOR PURE ODORANTS, the entire contents of which are hereby incorporated by reference.

Bilateral aroma testing may, but need not, include comparison using a known absolute concentration of aroma for scientific validation. Instead, a preferred embodiment may comprise a relative comparison, in finding which nostril is sensitivity is greater or weaker. Some variation is normal, but a profound difference is likely significant baring identifiable medical reasons for the loss of symmetrical sensitivity.

Some people have been exposed to industrial chemicals or paints that have damaged their sense of smell. However such chemicals likely impacted both nostrils relatively equally since such damaging chemical exposure was equal. In addition, research indicates that a relative equal loss of the sense of smell for pure aromas on both sides, as compared to a normal detection level, might be indicative of other neurological diseases, such as Parkinson's disease which impacts both nostrils equally.

Thus, a need exists in the art generally for an inexpensive, easy to use, accurate and repeatable clinically significant device, system and method for detecting an asymmetric (left vs right) differential in the olfactory detection threshold of a patient and/or for testing a patient's general olfactory capability.

The present invention addresses these, among other, needs.

### BRIEF SUMMARY OF THE INVENTION

The present system is directed in various embodiments to devices and methods for testing a patient's olfactory capability and/or sensitivity. The devices and methods may be used to test and compare a patient's ability to detect and/or identify an aroma or odorant presented to each nostril. Alternatively, the devices and methods may be used to generally assess, without regard to nostril-to-nostril comparative results, a patient's olfactory ability. The device may comprise a disk that comprises a plurality of odorant compartments that may be progressively advanced as the testing for a subject odorant is completed. The device may comprise a passive airway and an active airway, wherein only one nostril receives aroma-infused air while the other nostril receives atmospheric air. In other cases, the device may present aroma-infused air to both nostrils at the same time.

### BRIEF DESCRIPTION OF THE DRAWINGS

FIG. 1A illustrates a patient using a device of the present invention.

FIG. 1B illustrates an aroma media disk of the present invention.

FIG. 2A illustrates a front view of an embodiment of the present invention.

FIG. 2B illustrates a side view of the embodiment of FIG. 2A.

FIG. 3 illustrates an exploded view of one embodiment of the present invention.

FIG. 4A illustrates a top view of a lower shell of the present invention.

FIG. 4B illustrates a perspective view of the lower shell of FIG. 4A without nasal assembly.

FIG. 5 illustrates a perspective view of the lower shell of FIG. 4A.

FIG. 6A illustrates a front view of the rotational element of the present invention.

FIG. 6B illustrates a bottom perspective view of the rotational element of FIG. 6A.

FIG. 6C illustrates a to perspective view of the rotational element of FIG. 6A.

FIG. 6D illustrates a side view of the rotational element of FIG. 6A.

FIG. 6E illustrates a front view of the rotational element of FIG. 6A.

FIG. 7 illustrates an exploded view of the aroma media disk of the present invention.

FIG. 8A illustrates a top perspective view of the fin disk of the present invention.

FIG. 8B illustrates a bottom view of the fin disk of FIG. 8A.

FIG. 8C illustrates a bottom perspective view of the fin disk of FIG. 8A.

FIG. 8D illustrates a side view of the fin disk of FIG. 8A.

FIG. 9A illustrates a top perspective view of the aroma media disk and fin disk of the present invention.

FIG. 9B illustrates a bottom view of the aroma media disk of the present invention.

FIG. 9C illustrates a bottom view of the fin disk and media disk of the present invention.

FIG. 9D illustrates a top view of a top view of the media disk of the present invention.

FIG. 9E illustrates a side view of the fin disk of the present invention.

FIG. 10 illustrates a cutaway front view of the present invention.

FIGS. 11A-11C illustrate front cutaway views of the present invention.

FIG. 12A illustrates a perspective view of the upper shell of the present invention.

FIG. 12B illustrates a top view of the upper shell of the present invention with nasal assembly.

FIGS. 13A and 13B illustrate front cutaway views of an embodiment of the present invention.

FIG. 14 illustrates a rear cutaway view of an embodiment of the present invention.

FIG. 15A illustrates an embodiment of an aroma disk of the present invention.

FIG. 15B illustrates use of the aroma disk of FIG. 15A.

FIG. 15C illustrates adhesive patches to cover perforated aroma sources of the aroma disk of FIG. 15A.

FIG. 15D illustrates sealing a perforated aroma source of the aroma disk of FIG. 15A.

### DETAILED DESCRIPTION

While the invention is amenable to various modifications and alternative forms, specifics thereof are shown by way of example in the drawings and described in detail herein. It should be understood, however, that the intention is not to limit the invention to the particular embodiments described. On the contrary, the intention is to cover all modifications, equivalents, and alternatives falling within the spirit and scope of the invention.

The present system is directed in various embodiments to devices, systems and methods for detection, evaluation and/or monitoring olfactory dysfunction by measuring and determining the patient's olfactory detection threshold for the left and the right nostril. More specifically, the present invention relates to devices, systems and methods for detecting an asymmetric differential in a patient's relative olfactory detection threshold (left vs right nostril) which, when present, may be used as a device to detect, diagnose and/or monitor olfactory deterioration resulting from Alzheimer's disease. Alternatively, general olfactory deterioration may be tested and detected using the present invention.

### Definitions

As used herein, “symmetric” or “symmetrical” means that there is not a significant differential in the subject patient's ability to detect and/or identify odors between odors administered and/or inhaled into the patient's left nostril vs. the patient's right nostril as measured by the olfactory threshold determined for each nostril.

As used herein, “asymmetric” or “asymmetrical” means that there is a significant asymmetry or differential in the subject patient's ability to detect and/or identify odors between odors administered and/or inhaled into the patient's left nostril vs. the patient's right nostril as measured by the olfactory threshold determined for each nostril.

“Hyperosmia” is defined as increased olfactory acuity, or a decreased threshold for detecting odors, and may be symmetric or asymmetric as those terms are defined herein. Various embodiments of the present invention may detect and/or monitor hyperosmia or treatments therefore.

“Hypoosmia is defined as diminished or decreased olfactory acuity, or an increased threshold for detecting odors, and may be symmetric or asymmetric as those terms are defined herein. Various embodiments of the present invention may detect and/or monitor hypoosmia or treatments therefore.

“Anosmia” is defined as the inability to recognize odors and may be symmetric or asymmetric as those terms are defined herein. Various embodiments of the present invention may detect and/or monitor anosmia or treatments therefore.

“Dysosmia” is defined as the abnormal sense of smell and may be symmetric or asymmetric as those terms are defined herein. Various embodiments of the present invention may detect and/or monitor dysosmia or treatments therefore.

“Olfactory dysfunction” is defined herein as a patient with a disorder and/or condition with one or more of the following: hyperosmia, hypoosmia, anosmia, and dysosmia. The olfactory dysfunction may be symmetric or asymmetric as those terms are defined herein.

“Pure odorant”, also referred to equivalently as “pure aroma” is defined as substances including molecules and/or compounds which principally stimulate the olfactory cell receptors associated with the olfactory nerve, aka the first cranial nerve, and that do not trigger or excite the trigeminal nerve associated with the fifth cranial nerve. A non-exhaustive and categorized listing of pure odorants follows:

The pure odorant Spice family comprises:

Cinnamon;

Clove;

Vanilla;

Nutmeg; and

Allspice.

The pure odorant Food family comprises:

Peanut/Peanut Butter;

Coffee;

Cocoa;

Apple;

Almond (bitter); and

Strawberry.

The pure odorant Herbal family comprises:

Peppermint;

Spearmint;

Wintergreen;

Allspice;

Parsley;

Sage;

Turmeric;

Thyme;

Basil;

Dill weed;

Caraway;

Anise;

Fennel;

Mace;

Palmarosa; and

Patchouli.

The pure odorant Floral family comprises:

Rose;

Lemongrass;

Rosemary;

Lavender;

Lilac;

Violet; and

Origanum.

The pure odorant Citrus family comprises:

Orange;

Tangerine;

Lemon;

Lime;

Mandarin;

Grapefruit;

Bergamot and Petitgrain.

The pure odorant Wood and Resin based family comprises:

Eucalyptus;

Juniper berry;

Pine;

Tea tree;

Spruce;

Ho wood;

Cypress;

Cedar;

Birch;

Fir;

Cajeput;

Camphor;

Cassia;

Citronella;

Clary;

Copaiba;

Elemi;

Hydacheim;

Litsea; and

Niaouli.

“Pure odorant detection threshold” is defined as the point at which the concentration of pure odorant molecules saturate the olfactory nerve to the extent that a cognitive reaction first takes place. At this point, the subject patient is able to express that he or she is smelling something, but not necessarily able yet to identify the aroma by name. The pure odorant detection threshold may be found to be asymmetrical, i.e., significantly different as between the nostrils, indicating olfactory dysfunction. Alternatively, the pure odorant detection threshold may be found to be symmetrical between the tested nostrils.

“Pure odorant identification threshold” represents a slightly longer latent period than the “pure odorant detection threshold” as it is defined as the point at which the patient is able to actually identify the pure odorant by name, indicating that cognitive processing has occurred.

“Odorant” is defined herein as a compound that does trigger the trigeminal nerve.

“Odorant detection threshold” is defined as the point at which the concentration of odorant molecules saturate the olfactory nerve to the extent that a cognitive reaction first takes place. At this point, the subject patient is able to express that he or she is smelling something, without necessarily identifying the aroma. The odorant detection threshold may be found to be asymmetrical, i.e., significantly different as between the nostrils, indicating olfactory dysfunction. Alternatively, the odorant detection threshold may be found to be symmetrical between the tested nostrils.

“Odorant identification threshold” is defined herein as the point at which the patient is able to actually identify the odorant by name, indicating that cognitive processing has occurred. The odorant identification threshold may or may not be symmetrical between the nostrils.

“Effective amount” of the odorant or pure odorant is defined as the amount of pure odorant required to infuse the aroma airway passage during operation of the various devices, systems and methods of the present invention sufficiently to enable a patient to smell the pure odorant, i.e., when saturation of the olfactory nerve is sufficient to enable the reaching of the pure odorant detection threshold for the patient and nostril being tested.

“Clear air”, also referred to as pure air, is defined as air that does not comprise the odorant used in the inventive embodiments of the present invention. Preferably, clear air comprises air that is substantially uncontaminated by any odorant, including pure odorants. Clear air may comprise ambient air, i.e., atmospheric air, either filtered or unfiltered, or air that is provided from a clear air source such as an air tank or nebulizer and/or from a mechanized powered air pump as is well known in the art.

“Reset Period” is defined as a rest time between presentations or introductions of odorants or pure odorants, known reset periods are in the range of 90 seconds.

Aroma detection testing is done for many reasons and in a number of ways to accomplish various purposes, including diagnostic medical tests. Odor identification for example, comprise a common olfactory function that is tested. Numerous odor identification devices and methods have been developed to screen for what might be called a “scent memory function”. This sort of testing metric might be called “Tell me what you smell?” This corresponds to the presently defined “pure odorant identification threshold”. This endpoint for testing has several serious problems, not the least of which is the required inclusion of cognitive identification of the aroma or odor presented.

Pure aroma olfactory detection threshold response time is of particular clinical interest, especially when done bilaterally. In other words, the same aroma sensing test is preformed separately on each nostril and the test results compared, left nostril vs right nostril. This is done without aroma identification even being required. Perhaps the metric could be called, “Tell me when you smell something.”

A Baseline of Pure Air Vs Aroma Laden Air as a Testing Metric

Concentrations of a single aroma may be gradually increased in unscented air until cognitive awareness of the presence of the aroma is noted. The distance between the aroma source (a spoonful of peanut butter) and the nostril may be measured as a diagnostic metric upon the patient first noting the peanut butter aroma. “With your eyes closed and one nostril covered, tell me when you first smell the aroma, which I am slowly moving closer to your nose along the ruler”.

Alternatively, the time interval can be measured in seconds between aroma presentation to the subject, who is inhaling through an aroma presentation device, until the time interval ends upon cognitive notice that an aroma was noted. “Tell me the second you smell the aroma”. Time in seconds is the metric recorded, indirectly measuring the level of odor concentration of aroma required to trigger cognitive notice.

Similarly, the number of breaths taken from the point of first introduction or presentation of the aroma until cognitive notice by the subject is a valid metric indirectly scaling the aroma detection ability of the nostrils.

The aroma presentation device might emit an audible tone when the exhalation portion of the breathing cycle is taking place to help the testing personnel note the precise number of breaths taken during the presentation of the aromas, in this mode of testing. The number of breaths taken before cognitive notice of the aroma is also an indirect measurement of the concentration of aroma required to get cognitive notice, which measures olfactory function.

Finally, the metric of the absolute concentration of an increasing aroma level required for cognitive notice, (as measured by an electronic nose module with a digital readout), may be used in the pure air vs aroma air laden air model of bilateral olfactory testing. A plurality of sample of one pure aroma with a spectrum of dilutions might be presented with the smallest concentration detectable ramped up in small steps to a stronger concentration might be used with first one nostril and then the other to detect the weaker nostril.

The metric of single aroma might be considered comparing no smell in the air as a baseline compared to a slowly increasing concentration of pure aroma infused in clear room and recording some definable metric used for clinical comparison between the nostrils. All of these metrics indirectly score olfactory function for detection of pure aromas.

Cascading Aromas, an Preferred Alternative Aroma Detection Screening Metric;

Cascading a sequence of a plurality of pure aromas without a pure air reset period between aromas also scales the relative olfactory thresholds of the nostrils and has distinct advantages. The aromas may be presented manually where the aroma device is advanced to present a fresh aroma as the previous aroma is consumed or the device may be mechanically spring loaded, where the aromas are changed at the push of a button by the test subject during the screen. Alternatively, more than one device may be used to present the more than two odorants, or pure odorants to the patient.

The olfactory function of dissonance is far faster than “resetting” with a time delay, such as is required to reach a “no aroma detection condition” using clear air in preparation for a next aroma test event. The human olfactory latency period between having sensed a first aroma and being able to switch to sense a second aroma is far faster than going from aroma laden air to clear air.

The ability to ignore a first aroma and fully concentrate on a second aroma is of significant utility in using a cascading pure aroma threshold testing method. This method uses a metric of “rapid sequential aroma scoring”. “Cascading” a plurality of pure aromas, presented one immediately after another, ignores the currently required 90 second reset period between aroma presentations. This method speeds up the overall bilateral aroma testing process dramatically, as well as reducing scent latency issues.

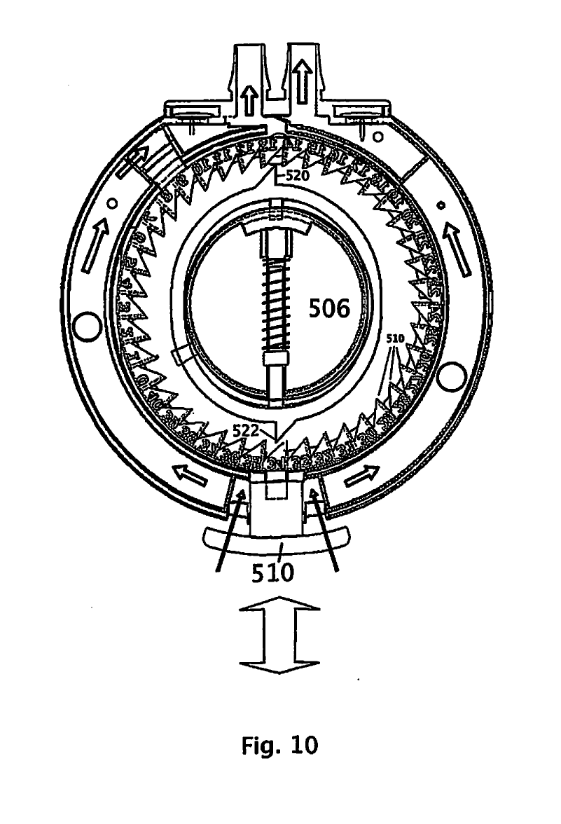

Aroma presentation devices without an aroma chamber, such as the one presented in FIG. 10 and higher, reduce the time spent breathing aroma infused air and allow an interesting plurality of aromas to fade in and out quickly.

The longer the latency period of one nostril, the weaker the sense of smell in that nostril. What we seek to detect is a significant relative deficit, particularly on the left side rather than an absolute value.

Theoretically, and according to previous clinical testing, the longer the period of time between exposure to aroma infused air and cognitive notice of that aroma, the worse the olfactory deterioration is in a particular nostril. The more time that is required to note the aroma, or a higher concentration of aroma required to trigger cognitive notice of that aroma, when tested bilaterally indicates relative olfactory deterioration of the two sides. More breaths equal more time and relative olfactory deterioration of the nerves associated with one nostril.

The memory function of an aroma, once cognitively recognized, (not necessarily named) remains in the olfactory “Que.”, until replaced by a subsequent aroma. It is thus, very hard to concentrate on smelling more than one aroma at a time. The new aroma replaces the previous aroma in our active olfactory memory.

“Olfactory aroma memory latency”, presents potential issues using a single aroma vs the clear air (air with no detectable aroma) mode of testing. It is hard to forget the recently presented aroma when only compared with theoretically clear unscented room air, making us hypersensitive to any latency of the first aroma. Unscented air in that method of testing must be used to purge the olfactory nerve and the aroma presentation device of latent aroma in preparation for the next testing event. Olfactory aroma latency is defeated by cascading a plurality of at least two pure odorants without a reset period.

A “reset” of the olfactory nerve may be defeated by even the slightest hint of the recently presented aroma. Aroma which is still lingering in the air or clinging to the airways of the aroma presentation device or the nostril can easily contaminate otherwise clear room air and spoil the sensitivity of the test. This is really the downfall of a one aroma screen.

While the recently presented aroma may be very faint after a reset period, the human sense of smell in some people is amazingly strong. The aroma latency issue thus creates a potential problem in obtaining accurate bilateral olfactory threshold sensitivity data with a clear air baseline sort of test.

Faint mechanical latency and human aroma memory latency issues, when using a handheld aroma presentation device, may be successfully overcome by “cascading” a new pure aroma immediately after another without using a clear air “palate cleansing” interval between aroma testing events. As soon as a first aroma is detected a subsequent aroma is strongly presented causing the first aroma to fade from memory without the problematic and time consuming step of clearing the olfactory “palate” with clear air.

The Cascading Aromas Device

The described hand held aroma detection device present a much more likely commercially viable embodiment of bilateral aroma testing for widespread clinical use than the larger afore mentioned desktop units or peanut butter and ruler as an apparatus. The handheld device, potentially removably clipped to a plaque on the wall of an examining room presents the notion of aroma testing being suitable for widespread, general clinical use as a neurological screening tool.

However, the aroma presentation device and aroma media disk might be disposable, which has the advantage of using a sterile device for each patient, having a fresh device would eliminate latent aromas and the cost of a disposable screening device might be commercially more profitable than reusing it for multiple patients.

A multiple aroma cascade presentation device might be designed such that the test is self-administered. The person being tested would simply hold the device comfortably up to their nostrils, close their eyes and press a button on the handheld device to automatically advance to the next aroma immediately upon noting the current aroma being presented to them. One first nasal cannula would present an aroma while the other second cannula would provide clear air. The device might be symmetrical such that it only need be taken away from the nostrils and reversed to make the second nostril the one presented aroma while the first nostril receives fresh air.

A series of tones could signal the starting point and ending point of the plurality of aroma presentations. A distinct battery-powered audible tone, such as a dinging sound, is sounded when the first aroma is presented and when the last aroma is noted. The tones are to allow time interval measurement by the clinical staff members as opposed to counting breaths.

A distinct battery-powered “exhalation tone or honking noise” could indicate that a breath has been exhaled. The handheld aroma presentation device emits an audible tone during exhalation to make the testing personnel aware of the breathing pattern of the subject.

A multiple chambered pure aroma or pure odorant cartridge may be mechanically spring loaded to advance in only one direction, one aroma chamber at a time. The multi cavity aroma media is rotated into alignment with a single airway communicating with the nostril, the device automatically advancing to the next aroma sample when actuated by pushing a control button.

A multiple chambered pure aroma or pure odorant cartridge may be mechanically spring loaded to advance in only one direction, one aroma chamber at a time. The multi cavity aroma chamber is rotated into alignment with a single airway communicating with the nostril, the device automatically advancing to the next aroma chamber when actuated by pushing a control button.

A first aroma is presented and the subject is told to take 5 breaths and then push a button conveniently located on the hand held aroma presentation device. A tone may be emitted by the device as it advances to the second aroma and a visual readout would confirm the aroma chamber in alignment with the airway for the test administrator to observe.

Upon advancing sequentially though all the aromas, back to first aroma, pushing the button to indicate that the first aroma (and last) was noted, a discrete audible tone indicates that the first (and last) aroma has been noted. The time sequence is stopped at that instant, the period in seconds between tones is the metric recorded for that nostril.

The timer may be a computer device, stop watch or a timer disposed and integrated into the hand held aroma presentation device.

Alternatively, the aroma presentation device might be manually adjusted to present one aroma at a time for the counting of breaths to the point of noticing a fresh aroma. The same device might also contain a plurality of aroma chambers such that an aroma identification test with more than 20 aromas supported might be done with the device.

The number of seconds can be counted from the first breath taken through the plurality of aromas until the first aroma is recycled to the last aroma, which is the stopping point of the test. For laboratory testing purposes a simple gas flow meter might be placed in line with the air inlet port to confirm that a uniform flow of air is being inhaled for each nostril being tested.

The number of seconds can be counted from the first breath to the breath where the fresh aroma is noted. For laboratory testing purposes a gas flow meter might be placed in line with the air inlet port to confirm that a uniform flow of air is being inhaled for each nostril being tested.

Multi-Aroma Chambered Devices

A hand held aroma presentation device might have a plurality of aroma chambers, which can be mechanically rotated or selectively re-aligned in a way as to present an open pathway for only one aroma at a time. Such a hand held screening tool is a very desirable apparatus.

Aromas are presented sequentially while closing access to all other aroma chambers. Such a device would speed up the testing procedure dramatically and reduce or eliminate significant aroma latency issues.

The person being tested would inhale through the aroma testing device to bring a plume of aroma infused air through only one of the plurality of aroma chambers. The aroma passing through the device air pathways into a nostril specula are presented directly into the test subject's nostril.

The improved hand held aroma presentation device described herein, might have 20 or more separate aroma chambers. Each chamber will selectively rotate in line with one airway port which is in fluid communication with the nostril of the person being tested. Each aroma chamber has a felt like pellet or disk held in place that holds a drop or more of aroma fluid. The active airway draws air through, or near to, the absorbent pellet or disk to infuse pure ambient air with an aroma.

The clinical employee or even the person being tested simply rotates an aroma presentation selector to align the single airway with the next aroma in sequence. The device is advanced immediately upon the test subject noting a fresh aroma was detected. When the number of screening events required are completed the cumulative elapsed time or number of breaths taken is recorded for each nostril and compared for a clinically significant difference.

The same device may be used in scent identification where the aromas are numbered such that the testing personnel can supervise the test as the subject checks off one of 4 or 5 possible aromas they smell through the device. The device may then be reversed to check the other nostril as 20 or more aroma are presented.

The aroma chamber device may be spring loaded with a coil type spring, a small motor or other mechanical device which upon the button being pushed advances the aroma chamber to the next station. Ratcheting gears known to the art may be employed to insure that the aroma chamber only advances in one direction. The audible tones may be electronic or mechanical with one tone to denote each new aroma as it is presented and a second distinctive tone is emitted at the presentation of aroma #1 when it appears as the final aroma.

Each multi-cavity aroma cartridge comprises separate chambers for holding at least two odorants or pure odorants, one odorant or pure odorant per chamber, but may hold 20 or more aromas comprising odorants or pure odorants.

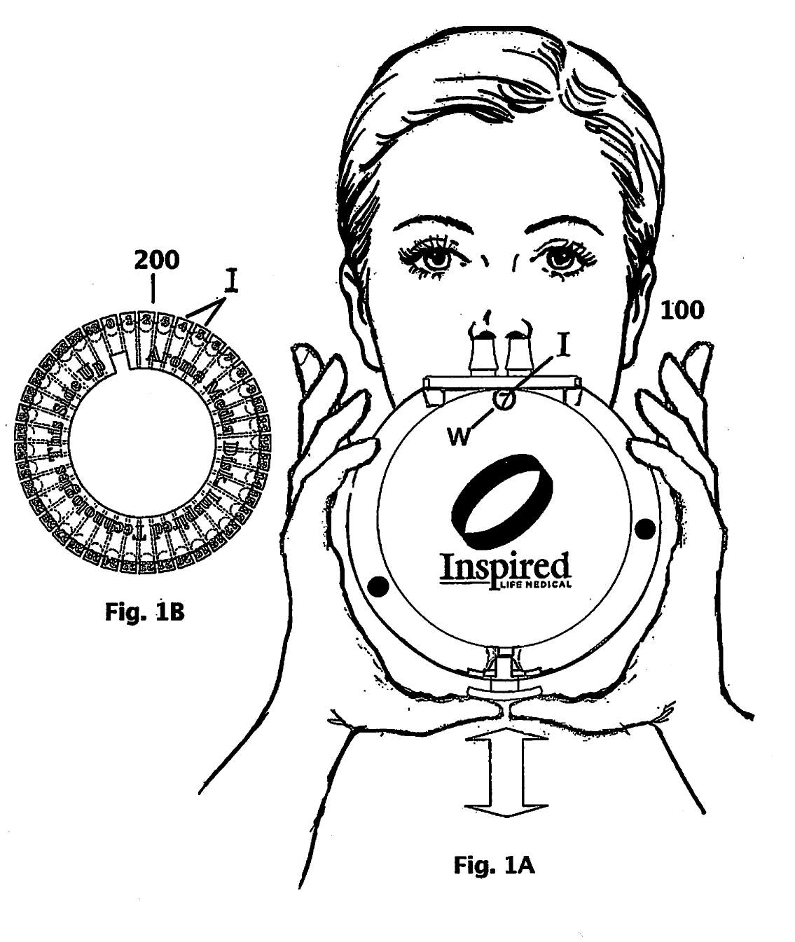

FIG. 1A illustrates one embodiment of the present invention in use, an assembled cartridge 100 with the testing administered by a 3^{rd} party, e.g., a health care professional. It is also an object of the present invention to self-administer the testing in some cases. FIG. 1B provides an illustration of one embodiment of an aroma media disk 200 of the present invention, with a plurality of aroma compartments 202 arranged around the disk and a central aperture. Note the indicia I, e.g., numbering, around the circumference of the aroma media disk 200 that appears in indicia window W defined through openable door 602 in FIG. 1A. This indicates that aroma “7” is currently being tested which corresponds with an aroma key to maintain masking of the identification of the aroma from the patient's perspective while maintaining testing fidelity for the administrator of the test.

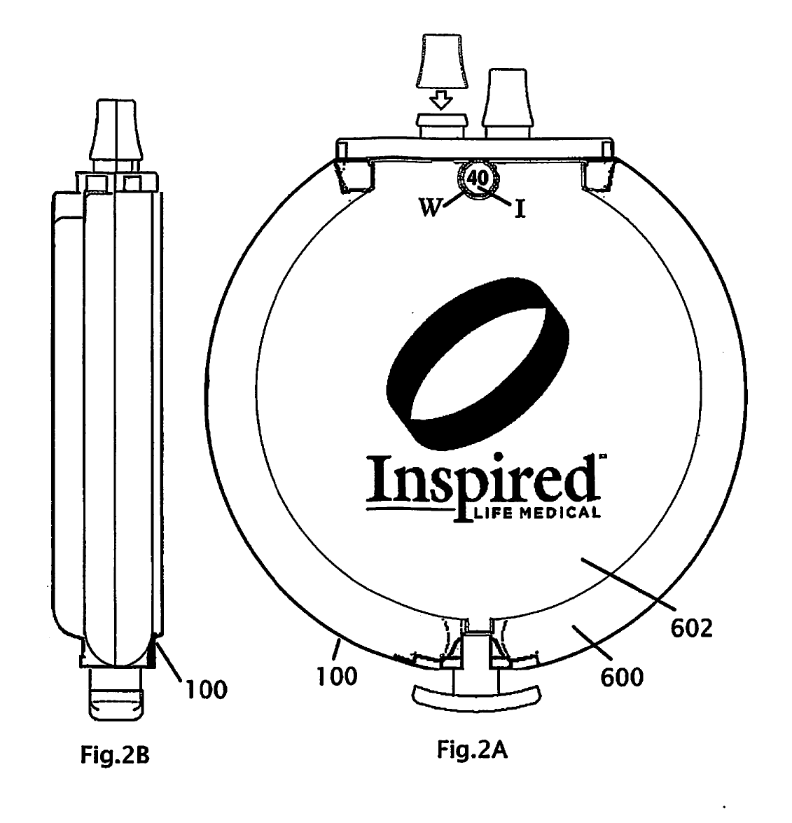

FIGS. 2A and 2B illustrate cartridge 100 in front and side view, respectively.

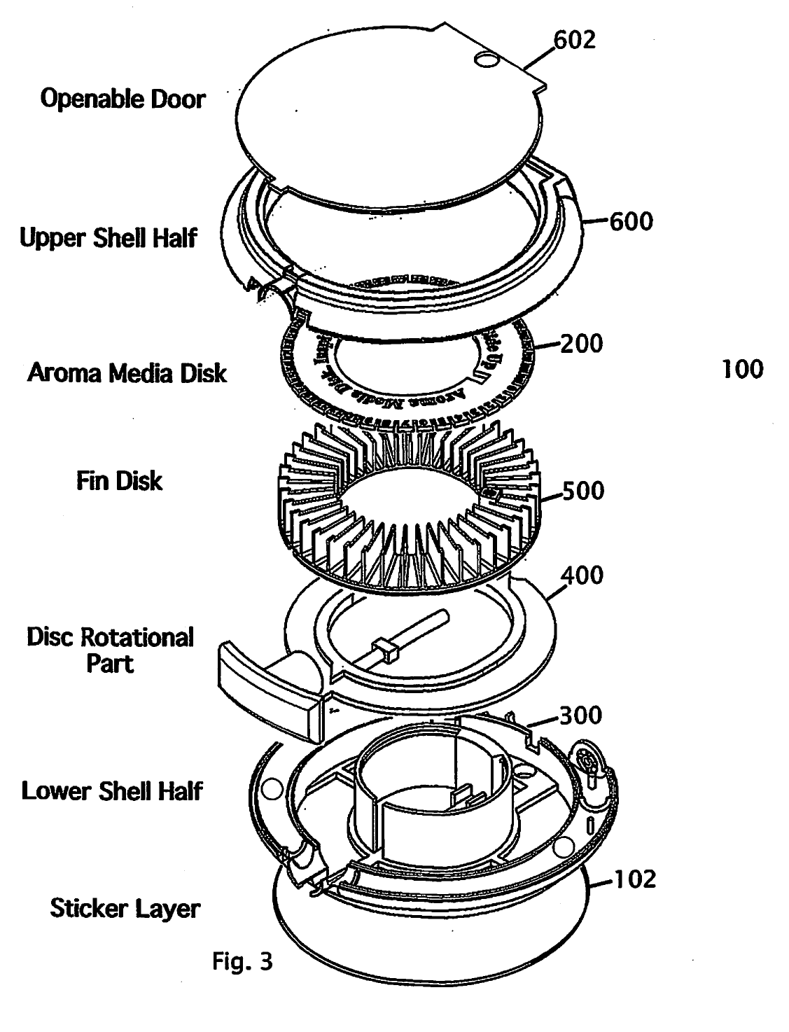

FIG. 3 shows the cartridge 100 in exploded view. Thus, an optional sticky layer 102 is provided and attached to the lower shell 300 which is in operative engagement with a rotational element 400. Fin disk 500 is received by the rotational element 500 and the aroma media disk 200 is, in turn, provided in operative engagement with the fin disk 500. Upper shell 600 engages with lower shell 300 and further comprises an openable door 602.

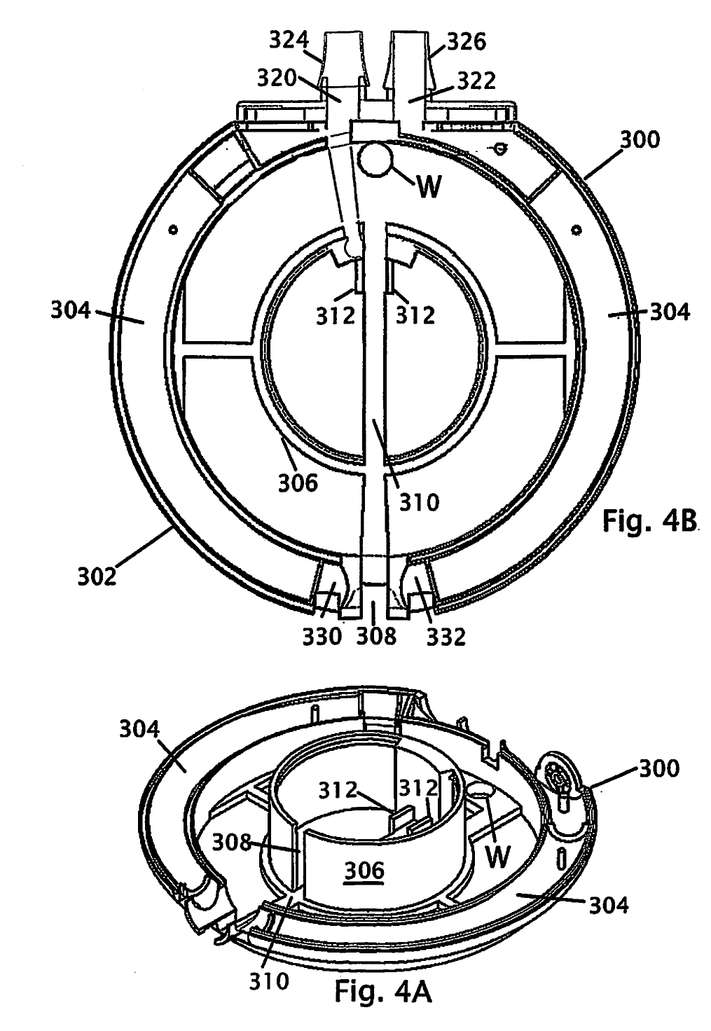

FIGS. 4A and 4B illustrate the lower shell 300 in top and perspective view, respectively. Lower shell 300 comprises generally a housing 302 with first and second circumferential concave regions 304, 304′ that form one side of a first and a second airway channel, e.g., passive and active airways, when the cartridge 100 is assembled. A slotted central cylinder 306 is provided and that surrounds a center point of the lower shell 300. Slot 308 runs down through the height of central cylinder, terminating at the bottom of central cylinder. A raised support element 310 runs through the slot 308 and extends radially across the central cylinder 306, terminating within central cylinder 306. Two raised alignment supports 312, 312′ are provided at the terminal end of the raised support element 310. Central cylinder 306 is configured to engage or be received within central aperture of aroma media disk 200.

Circumferential concave regions 304, 304′ are interrupted by a nasal assembly at the top side and an inlet region at the bottom side. Nasal assembly comprises a first and a second conduit 320, 322, each with nasal cannula 324, 326 attached which are preferably removable and replaceable. The first and second conduits 320, 322 are in operative communication with first and second airway channels when cartridge is assembled, respectively. Inlet region provides separated first and second air inlets 330, 332 in fluid communication with the first and second airway channels, respectively which are discussed further below.

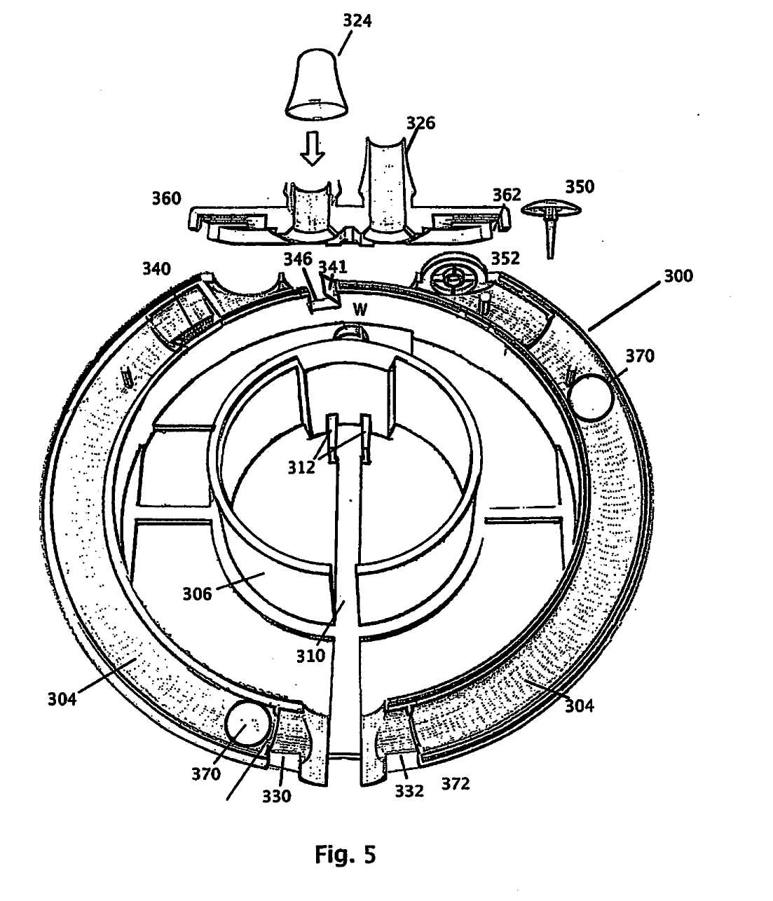

FIGS. 4A and 5 provide further detail for lower shell and the resulting airflow in assembled cartridge 100. Thus, airflow is actuated by inhalation at the nasal assembly. Airflow progresses through first inlet 330 through the first airway defined in part by first circumferential region 304. Airflow in first airway is diverted radially inward through channel to move through and over an aroma source to become infused as best seen in FIG. 4A, then rediverted upward through channel 346, which as a result of tab 341 which separates the first and second airways, keeps the infused air within the first airway only. Tab 341 may be in certain embodiments removable and/or insertable or replaceable to modify the airway passages from unmixed to mixed or from mixed to unmixed airflow. Tab 341 is located at a point that is upstream from the nasal conduits 324, 326 and downstream from the currently active aroma source 202 that is rotated into in the active airway passage. From channel 346, the infused air passes through first nasal conduit 320. This first airway is an “active” airway, i.e., infused with aroma.

The other nostril activates airflow through second inlet 332 which then progresses through second airway defined in part by second circumferential region 304′, continuing to pass through second nasal conduit 322. This second airway is “passive” in that no aroma or odorant is infused therein.

Exhalation airways 360, 362 are governed by one-way valves, see e.g., valve 350 seated in valve seat 352. Inhalation is also governed in part by one-way valves at 370, 372.

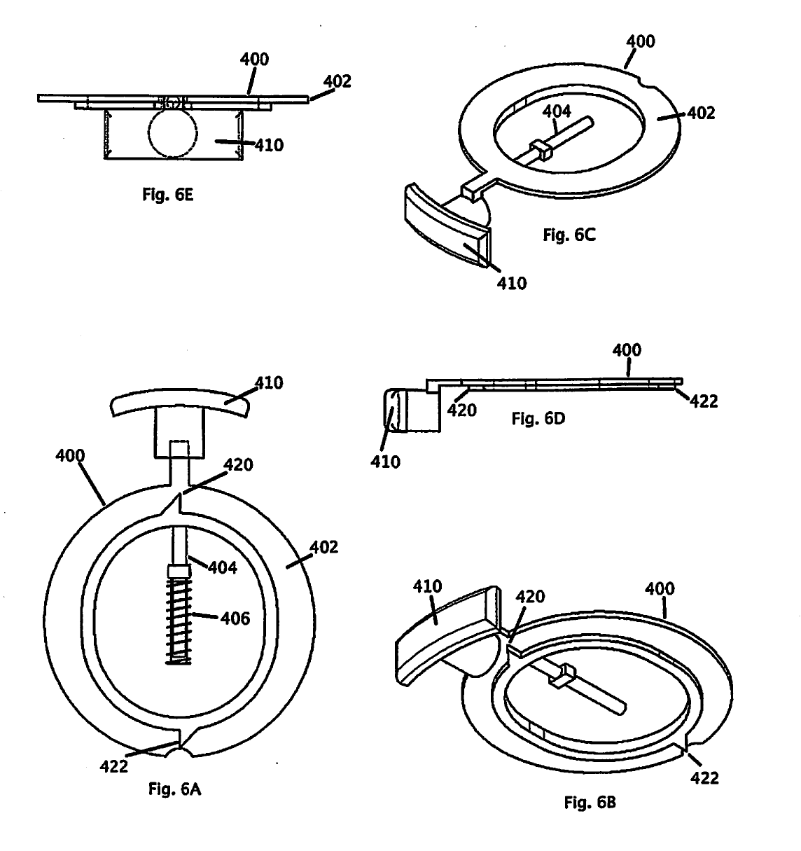

FIGS. 6A-6E show the rotational element 400 from various perspectives. A circular base is provided that fits around central cylinder of lower shell 300 and which supports an arm that 404 is spring loaded or biased, with a spring 406 located around arm 404 proximate the distal end of arm. The arm 404 is adapted to fit within the lower shell 300 along the raised support element 310 of lower shell 300, within the slot 308 of central cylinder 306 and within the two raised alignment supports 312, 312′. A push handle 410 for actuating rotation of the aroma media disk 200 is provided, wherein overcoming the spring bias allows the push handle 410 to be translated toward lower shell housing 302, with concurrent rotation of the aroma media disk 200. Two angled teeth, upper tooth 420 and lower tooth 422, are provided, each angled in the same direction and with same angle and on opposing sides of the circular base 402, the teeth 420, 422 extending radially toward an outer edge of the circular base 402.

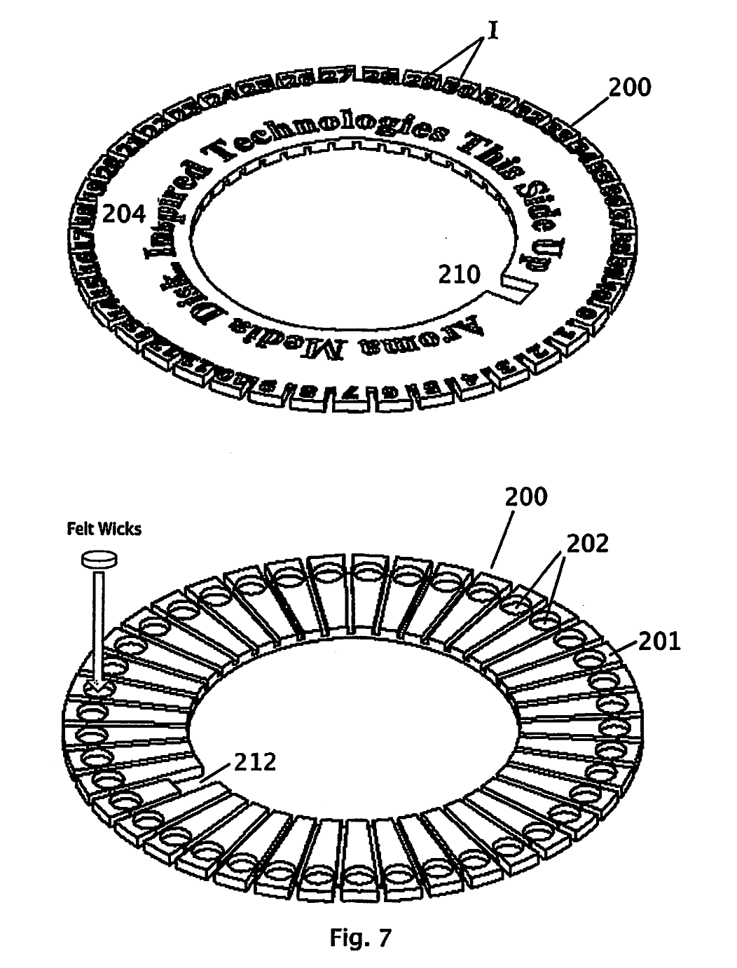

Aroma media disk 200 is shown in FIGS. 7A and 7B with a base 201 comprising a plurality of individual ordorant or aroma compartments 202 that may each comprise an odorant or aroma source within the specific odorant or aroma compartment, e.g., a scented felt pad or the like as shown. Indicia I, e.g., numbers may be provided on a sealing cover 204 that sealingly engages as shown to assist in identifying the specific aroma or odorant being tested. In some cases, selected chambers or compartments 202 may be blank. The sealing cover 204 and the base 202 may comprise keying slots 210, 212, respectively, that, when cover 204 and base 202 are sealingly engaged, align to assist in ensuring proper identification of the material within each compartment 202 when the aroma media disk 200 is installed in cartridge 100.

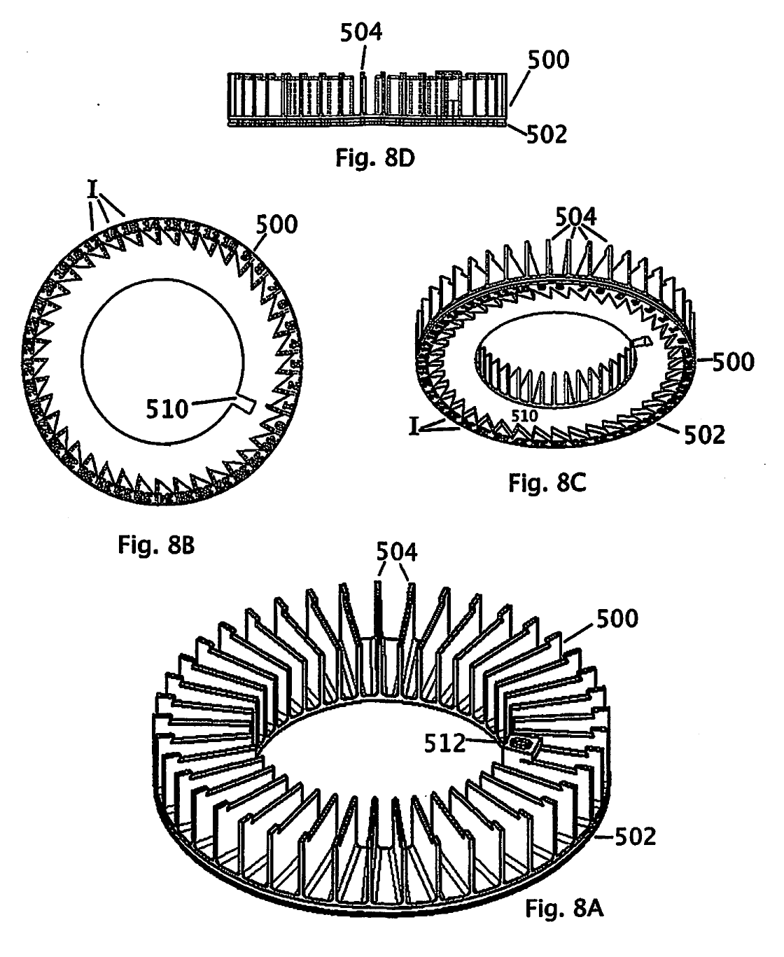

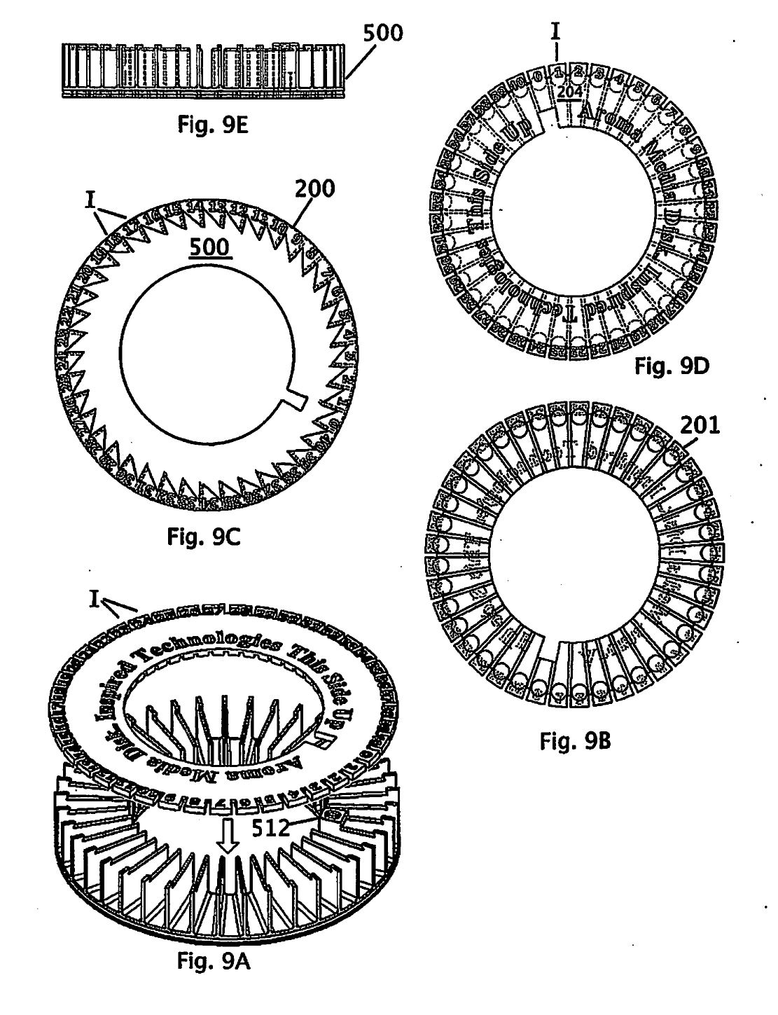

FIGS. 8A-8D and 9A-9D illustrate various views of fin disk 500 and in combination with aroma media disk 200 (FIGS. 9A-9D). Fin disk 500 comprises a base 502 with a plurality of spaced apart partitions or fins 504 rising away from a front side of fin disk 500 and arranged or configured to engage the plurality of individual compartments 202 of aroma media disk 200 so that one aroma in an odorant or aroma compartment 202 of media disk 200 is held between two adjacent partitions 504 of fin disk 500. Fin disk 500 further comprises a circumferentially arranged series of angled teeth 510 designed to interact with the rotational element 400 in a geared relationship with the two teeth 420, 422 of rotational element 400 to advance the media disk 200 one aroma compartment and aroma at a time. Note, as best seen in FIG. 9A, the key 512 located on the top of one of the plurality of partitions of fin disk 500 which, when placed into keyed engagement with the keying slots 210, 212 of media disk 200, provides keyed indication and identification of the first aroma presented to the patient at the nasal assembly.

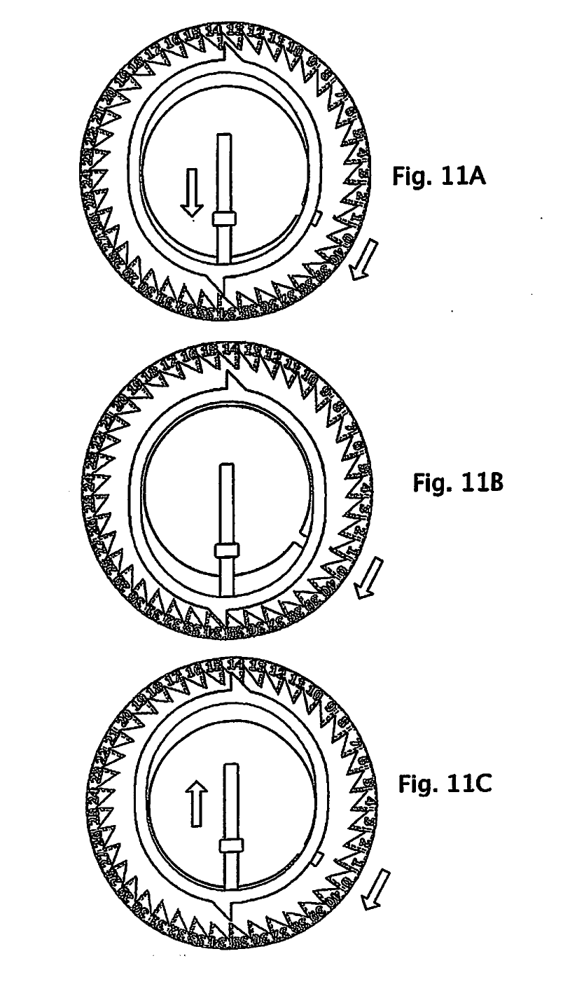

FIGS. 10, 11A-11C show the relationship between the two teeth 420, 422 of rotational element 400 and the angled teeth of fin disk 500. The angled teeth 510 of fin disk 500 are extending inwardly toward the angled teeth 420, 422 of rotational element 400. There are an odd number of teeth on fin disk 500 and an even number (in this case two) teeth on the rotational member 400 which may be out of phase with the fin disk teeth 510 by the distance of one-half of the width of a tooth. The upper and lower teeth 420, 422 of the rotational member 400 are half-way out of phase with the fin disk teeth 510 due to the geometry of the teeth and the bias of the spring which holds the teeth in engagement and again when the push handle 410 of rotational member is fully depressed. FIGS. 11A-11B provide a progression of advancement of the rotational member, fin disk and odorant compartments. As described, each advancement corresponds with a single odorant compartment moving into the active airway channel.

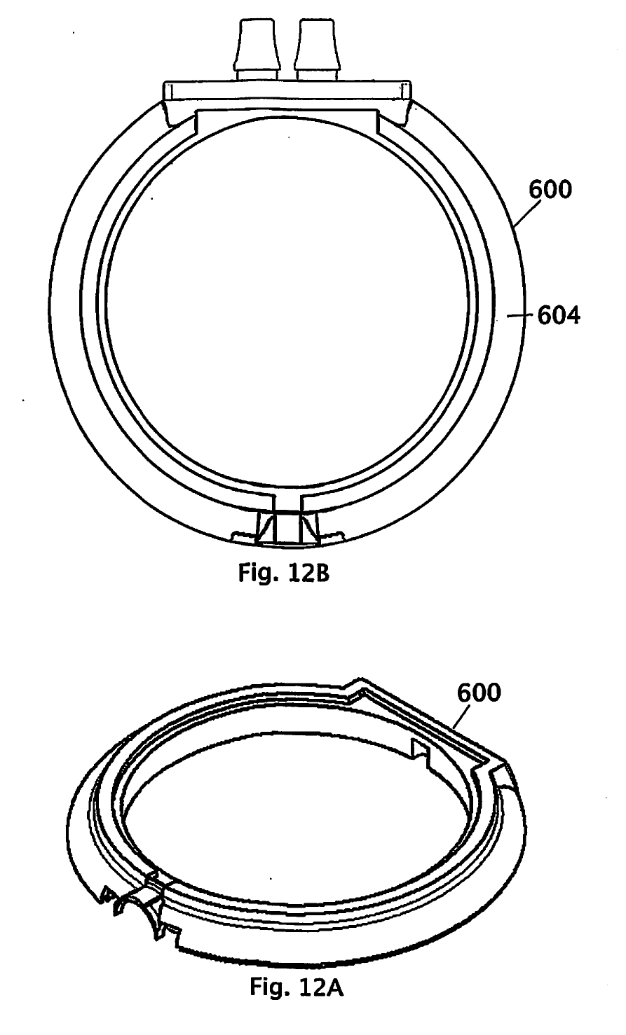

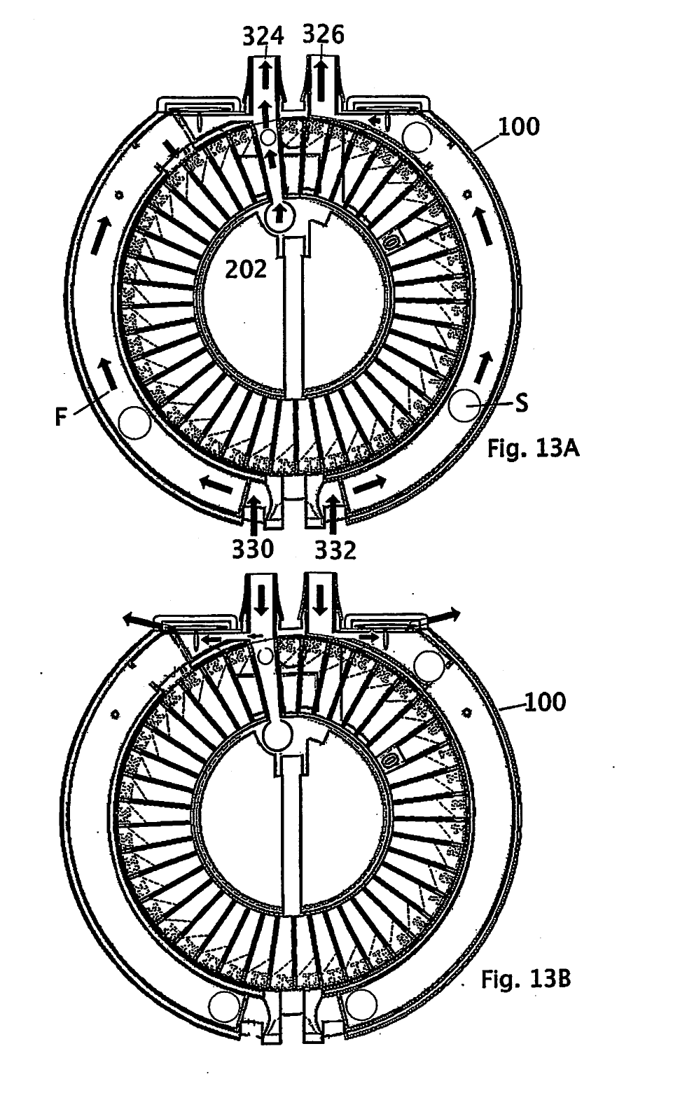

FIG. 12A provides a perspective view of the upper shell 600 which as shown in FIG. 13B operatively engages nasal assembly in combination with lower shell 300 as discussed above. Upper shell 600 provides a complementary circumferential concave region 604, 604′ to the circumferential regions 304, 304′ of lower shell 300 such that, when assembled, the two circumferential concave regions form the first and second airway channels. As will be discussed, each side of the formed passage is dedicated to one nostril in certain embodiments to provide an active airway infused with an aroma or odorant and a passive airway drawing from atmospheric air and, therefore, free of infused aroma.

The concept of separate airways is shown in FIGS. 13A and 13B wherein a first airway inlet 330 corresponds with a first airway F, and a second airway inlet 332 corresponds with a second airway S, wherein the first and second airways F, S are formed of the two circumferential concave regions of upper and lower shell 600, 300 when assembled and as described above. Similarly, first and second airway inlets 330, 332 are formed as a consequence of the engagement of upper and lower shell 600, 300. In the example, in response to patient inhalation at first nasal conduit 324, the first inlet 330 and first airway F pulls air from the atmosphere, through the first airway F and then is directed through and across or near one odorant or aroma compartment 202 comprising an aroma source therein, or which may comprise a blank. In this way, the air moving through the first airway is infused with the aroma source (or not infused if a blank). The second inlet 326 and second airway S is restricted to not move through an aroma source contained in an aroma or odorant compartment 202 and, therefore, is not infused with any aroma source, i.e., is clear air.

As discussed above, once the patient has completed testing with the subject aroma in the active odorant compartment, pressing the handle of rotational element advances the media disk 200 one odorant compartment 202 to present a new aroma or odorant.

When the entire battery of the plurality of aroma compartments has been completed for one nostril, then the device 100 may be rotated 180 degrees to engage the second nostril with the active airway passage for repeating the testing that was completed for the first nostril. In this way, the relative sensitivities of the patient's ability to detect and/or identify odorants or aromas may be compared on a left vs right nostril basis.

Returning to FIG. 5, tab 341 is the element that prevents the atmospheric air from reaching an odorant chamber on the active airway side of the device and prevents infused air from contaminating the passive clear or non-infused air on the passive airway side. Airflow encounters tab 341 and is redirected up to the relevant nasal passage 324, 326 and to the patient's nostril. If, however, tab 341 is removed, the airflow may be mixed such that both of the patient's nostrils may be exposed to aroma or odorant infused air. In some embodiments, this may be desirable to test general olfactory capabilities, without regard to left vs right nostril sensitivities.

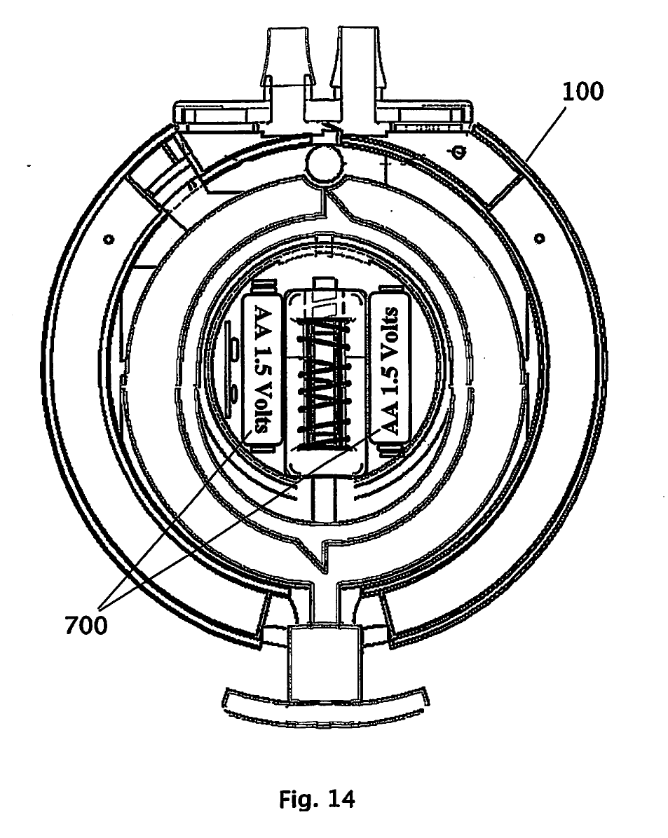

FIG. 14 provides an embodiment comprising battery powered elements 700, e.g., audible tones, lights and the like. Ultrasonic induced aroma sources may also be powered by battery.

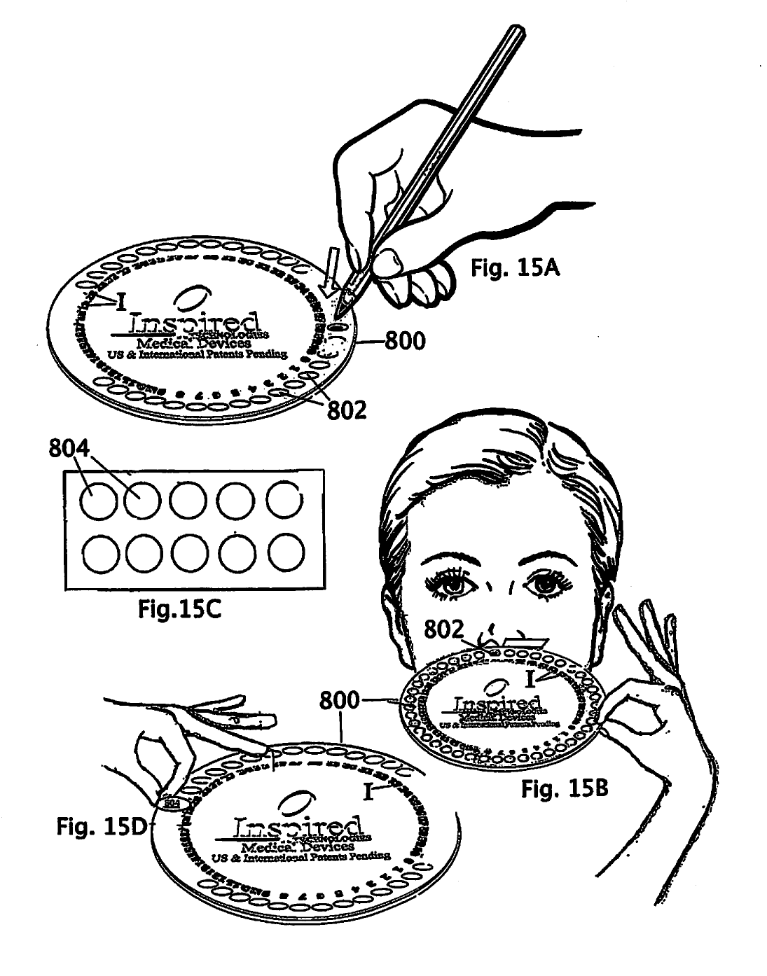

FIGS. 15A-15D provide an embodiment that simply uses a media disk 800 without further aid of an inhaling device. As shown, in FIG. 15A a series of covered aroma sources 802 are provided on a substrate, each source comprising an associated indicia I such as a number or the like. The next source 802 in the battery of testing may be perforated as shown at 802′ to allow the patient to access the aroma or odorant within as shown in FIG. 15B. Adhesive patches 804 or equivalent may be provided to cover each aroma source and that may be removed and/or punctured or perforated to access the aroma source to sniff. Once the test is completed for a specific aroma, that aroma source may be covered, or recovered by an adhesive patch 804 or equivalent as in FIG. 15D, and the next aroma source accessed to test.

Thus, this exemplary device may be used to present a more than one pure odorant in a cascading fashion, i.e., sequentially and within a reset period between presentations. Initially, a first pure odorant is made available for presentation to one nostril of a patient, wherein the patent inhales to activate the inhalation air flow pathing described above, and exhales into the device until a fixed number of breaths are reached, or a fixed time has elapsed or until the patient recognizes they have reached the pure odorant detection threshold, whereupon the relevant metric is observed and recorded. At this point, the multi-chamber pure odorant cartridge is advanced to the next pure odorant and the process is repeated and again if additional pure odorants are provided in the testing process and in the cartridge.

When the first nostril testing is completed, the same process is repeated with the second nostril. Ultimately, the results are compiled and the left nostril and right nostril data is compared for substantial differences between the left and right nostril data sets.

Note that the methods, devices and systems disclosed herein have utility for pure odorants in the measurement of bilateral pure odorant detection thresholds. These devices also may be used for any odorant, including pure or non-pure odorants. In other words, odorants that also stimulate the trigeminal nerve (non-pure) may also be used with the disclosed devices and methods. Further, as discussed above, general olfactory capability and sensitivity may be assessed without regard to comparative ability between nostrils.

In the case of non-pure odorants, the metric used will comprise an odorant identification threshold. Thus, the patient will be presented with the odorant-infused air as described above for pure odorant-infused air, and the next odorant sequentially introduced as soon as the patient identifies the first odorant by name. This identification point is defined herein as the odorant detection threshold. Preferably, the odorants' sequential presentation using the described devices and methods is done without a reset period between odorant introductions.

A computerized application and alternative embodiment of the method may be provided. Here, the data may be entered by hand into a spreadsheet previously created and saved within the memory of a programmable computing device, accessory or appliance such as previously described or may be automatically communicated by, e.g., a USB device as described herein that is connected to the testing device and in communication, i.e., wired or wireless, with the computing device. In addition, the computing device can control the testing device in terms of stopping and starting aroma presentations.

Moreover, a software database and testing protocol support application(s) may be used to achieve the testing described herein with any of the disclosed device and system embodiments of the present invention. The software database may be within individual computing devices and/or may be housed within a central server that is interconnected with individual computing devices that are located at testing sites. As illustrated, at least one central server is provided and in communication with at least one remotely located computing device. Central server(s) may be cloud-based which may permit controlled access from any internet connected device, preferably a secure account enabled internet connection is employed.

Thus, a programmable computing device for implementing the invention may comprise: a memory, wherein the application, including programmed instructions for running the test protocol embodiments described herein is stored and for storing test results; a processor operatively connected with the memory and which executes the application and associated programmed instructions; a display that may display the application, test data results for left and for right nostril trials, trial number, a timer and the final calculated results in terms of any differential between the left and the right nostril detection thresholds, or a differential between a previous baseline or population statistical average score, and the instant test score. The display is operatively connected with the processor and memory; and a transmitter and a receiver for operatively connecting, and communicating with, the central server. In this system, the testing results may be obtained at the testing sites and added, either manually or automatically as described herein, to the computing device for storage and possible transmission of the data to the central server.

When the testing procedure is complete, the test data may be sent, either automatically or upon prompting by the user, from the computing device at the associated test site to the remote central server. Central server comprises a memory for storing the received test data from the at least one computing device and associate test site(s) and for storing an algorithm for processing and analyzing the instant test site results; a processor for executing the programmed instructions within the stored algorithm; a transmitter and a receiver operatively connected with the at least one computing device whereby two-way communication with the at least one computing device is enabled. Central server's memory further comprises a database for storing all of the test results received from the at least one computing device which may be used to develop further refined and more robust statistical conclusions regarding relevant elements of the patient's medical history and the instant test data received from the at least one computing device for an individual patient and securely transmit the calculated disease risk score based at least in in part upon global data stored within the memory of the central server and reported to the local computing device. Robust encryption and security features may be employed to protect individual patient's privacy rights.

The algorithm of the central server may analyze the data received from the at least one computing device and, when analysis is complete, the central server may transmit an electronically secure summary of the testing results as a risk score as described above back to the computing device at the test site so that the user, i.e., a health care provider, can observe the results by, for example, a secure email sent to a predetermined email address.

In addition, a separate application or, alternatively, an internet browser supported client program may supply a checklist of a patient's pre-testing history and enable establishing of the patient's clinically acceptable baseline of nasal performance, including any relevant medical history factors such as structural or medical issues that may compromise the left or the right nostril/airway performance and/or efficiency. This baseline value may be incorporated into the above algorithm to provide a corrective factor that essentially treats any observed airway performance for the left and/or right nostril and associated airway as a variable that may skew the final results if not corrected. The database described above may also accept input of this data and incorporate it into the analysis phase to enable a corrected result to be calculated and typically securely communicated to the appropriate computing device and associated test site.

A combination of data types may be obtained using the devices and methods of the present invention, e.g., capturing the elapsed time between introducing aroma to the aroma airway passage and the detection thereof by the patient, the number of breaths required to detect the introduced aroma and/or the absolute concentration of odorant, or pure odorant, required to reach the olfactory threshold for each nostril. The data may be analyzed by the local computing device's application and/or analyzed remotely at the central server(s) as described above in order to determine the patient's odorant, or pure odorant, detection threshold.

Aroma sources as described herein may be in the form of a liquid held in an absorbent porous material such as a wick, stiff blotter slide or a cotton ball that is placed in the aroma chamber of the test apparatus. A viscous material such as peanut butter could be wiped onto a slide like element and inserted into the aroma chamber or the material supplied in a disposable portion package with removable seal top. Odorants, or pure odorants, may be used as discussed herein.

Such aroma/odorant diffusion devices are suitable to use as a cartridge that is inserted into the housing of the various devices as described herein. Such devices are refillable and may be filled with any essential oil. USB type ultrasonic devices emit little aroma when switched off. They may be used in the test devices of the present invention comprising, e.g., a single air chamber, thus reducing the complexity and parts required to manufacture such devices.

Managing Global Testing Data

No medical testing apparatus or method has diagnostic value without a convincing amount of data to document efficacy. The combination of the disclosed handheld aroma testing mechanism and aroma testing protocol with associated data, can be well managed to have dramatic potential for many purposes.

From the medical provider's point of view, there is an internet based web site containing the most recent information on the aroma scale test, provider account registration and the practice's data for its patients behind a secure login infrastructure.

The medical provider establishes an account for their practice that includes providing a physical clinic shipping address, phone numbers, credit card account information, a designated email account and other contact information.

A password protected “provider account” is thus set up to support the online purchase of Aroma Scale devices, consumable Aroma Cartridges, nostril airflow testing meters, etc. A unique Medical Provider Account identifier code is provided which is used in establishing secure patient file access. Provider accounts include an automatic payment system utilizing a credit card for paying for aroma scale reporting services and products.

A computer application that runs on iPad, iPhone, Android, Macs and PCs allows secure patient data files to be established and the data accessed by the medical provider.

Secure Patient files contain relevant medical history, nostril airflow data and dated aroma scale test data for any patient given the aroma score screening test. This account information is “HEPPA secure” and may require the use of unique patient identifier codes that only the doctor can correlate with any particular patient. Follow up aroma testing is added to the existing patient file along with date and relevant medical history information.

The computer application may be in the form of a stand alone “App” that is distributed free, by Apple Computer Company and various Android App Stores. Alternatively, a web based HTML5 App would be available offering similar functions running on common web browsers.

To begin using the Aroma Scale screening system a provider would set up a provider account, order aroma scale testing apparatus and train nurses who will administer the test in their clinics. Training may be done by using a DVD or viewing videos posted on line.

A database populated with numerous patient files would allow for “virtual” observational clinical studies. Further medical history information may be added periodically to updated data input forms, as helpful risk factors are proposed. Requests for database analysis reports would be fulfilled by the central computer database under the control of the Aroma Score Company.

An extensive database would provide candidates for drug studies since known early onset AD and similarly situated people who are not early onset AD would be searchable by locality. The demand for highly concentrated early onset AD patients who are local and willing to participate in clinical trials is a major obstacle for the drug industry, worldwide in doing clinical trials.

Screening a patient includes the protocol elements of:

1. Opening a new online data entry form using an application or web form. The data entry form requires every block of data requested be entered before going to the following page to insure complete files.

2. A medical history page includes, the provider account identifier code, the system automatically loads the date of the test, requires an entry that encrypts a novel patient identifier code, records date of birth, sex, weight etc., including relevant known medical history, nostril airflow numbers and the Aroma Scale raw data.

3. Upon completing the patient data entry form the nurse enters “Submit” to send the new patient file over the internet to the Aroma Score central server. This completes the test and data entry portion of the Aroma Score management method.

4. Upon receiving the patient data file over an internet connection, the central server adds the disclosed information to a central data base and correlates the instant data provided with all existing data on the server.

5. The server then uses a proprietary algorithm to generate an AD risk score for that particular patient considering all the medical history and testing data amassed globally.

6. The server then charges the medical provider's credit card a processing fee for providing an AD risk score. Follow-up Aroma Scoring processing might be free for registered patients to encourage participation with the program long term.

7. The server then sends the medical provider an email that include the encrypted patient identification code, the report lists known risk factors for AD and an Aroma Scale Risk Assessment.

8. The medical provider then uses the Aroma Scale Risk Assessment to assist in making a diagnosis or decision to do more AD risk testing. The diagnosis could include, planning a follow up aroma scale screen in the future, doing alternative testing to confirm or exclude incipient AD in case of a high risk assessment. The risk assessment may completely clear the patient, for the time being.

The present invention should not be considered limited to the particular examples described above, but rather should be understood to cover all aspects of the invention. Various modifications, equivalent processes, as well as numerous structures to which the present invention may be applicable will be readily apparent to those of skill in the art to which the present invention is directed upon review of the present specification.

What is claimed is:

1. A device for testing olfactory sensitivity to one or more aromas, the aromas comprising odorants and/or pure odorants, the device comprising:
a housing defining a first active airway channel and a second passive airway channel, the first active airway channel providing airflow consisting of aroma-infused air to a first nasal conduit and the second passive airway channel providing airflow consisting of atmospheric air to a second nasal conduit;
an aroma media disk keyed within the housing and comprising a plurality of selectable and circumferentially positioned aroma sources, wherein each aroma source is spaced apart and partitioned from adjacent aroma sources and wherein one aroma source at a time may be in an active position within the first active airway channel; and
a tab disposed between the first active airway channel and the second passive airway channel, wherein the tab is located downstream from the active aroma source and upstream from the first and second nasal conduits.

2. The device of claim 1, wherein there is no mixing of the aroma-infused airflow with the airflow through the second active airway before the airflows reach the first and second nasal conduits.

3. The device of claim 1, further comprising a rotational element operationally engaged with the aroma media disk configured to rotationally advance the aroma media disk within the housing and to advance the plurality of aroma sources to the active position within the first active airway channel one at a time.

4. The device of claim 1, wherein the plurality of aroma sources comprises at least one pure odorant.

5. The device of claim 1, wherein the aroma media disk further comprises indicia for each of the circumferentially positioned aroma sources.

6. The device of claim 1, further comprising a window defined within the housing that aligns with the active aroma source indicia.

7. The device of claim 1, the housing further comprising a central cylinder and wherein the aroma media disk comprises a central aperture, wherein the central cylinder of the housing is received within the aroma media disk's central aperture.

8. The device of claim 1, wherein the device is used to test the sensitivity of each nostril to the plurality of aroma sources.

9. The device of claim 4, wherein the device is used to test the deterioration of the patient's left olfactory lobe and, separately, the deterioration of the patient's right olfactory lobe.

10. A device for testing olfactory sensitivity to one or more aromas, the aromas comprising odorants and/or pure odorants, the device comprising:
a housing defining a first active airway channel and a second passive airway channel, the first active airway channel providing airflow consisting of aroma-infused air to a first nasal conduit and the second passive airway channel providing airflow consisting of atmospheric air to a second nasal conduit;
an aroma media disk keyed within the housing and comprising a plurality of selectable and circumferentially positioned aroma sources, wherein each aroma source is spaced apart and partitioned from adjacent aroma sources and wherein one aroma source at a time may be in an active position within the first active airway channel; and
a removable tab disposed between the first active airway channel and the second passive airway channel, wherein the removable tab is located downstream from the active aroma source and upstream from the first and second nasal conduits.

11. The device of claim 10, wherein there is mixing of the aroma-infused airflow with airflow through the second active airway before the airflows reach the first and second nasal conduits.

12. The device of claim 10, further comprising a rotational element operationally engaged with the aroma media disk configured to rotationally advance the aroma media disk within the housing and to advance the plurality of aroma sources to the active position within the first active airway channel one at a time.

13. The device of claim 10, wherein the plurality of aroma sources comprises at least one odorant.

14. The device of claim 10, wherein the aroma media disk further comprises indicia for each of the circumferentially positioned aroma sources.

15. The device of claim 10, further comprising a window defined within the housing that aligns with the active aroma source indicia.

16. The device of claim 10, the housing further comprising a central cylinder and wherein the aroma media disk comprises a central aperture, wherein the central cylinder of the housing is received within the aroma media disk's central aperture.

17. The device of claim 10, wherein the device is used to test the sensitivity of both nostrils at the same time to the plurality of aroma sources.

18. A system for testing a patient's olfactory sensitivity, comprising:
a base substrate comprising a plurality of sealed aroma sources, wherein each of the sealed aroma sources may be accessed to release the aroma source;
media for resealing the accessed aroma sources; and
indicia for each of the plurality of sealed aroma sources adapted to a key of the identity of each of the sealed aroma sources.

19. The system of claim 18, wherein the sealed aroma sources comprise pure odorants.

20. The system of claim 19, wherein the sealed aroma sources comprise non-pure odorants.
